(19) United States
(12) Patent Application Publication
(10) Pub. No.: US 2010/0144809 A1
(43) Pub. Date: Jun. 10, 2010

(54) NOVEL BENZAMIDINE DERIVATIVES USEFUL AS POTASSIUM CHANNEL MODULATORS

(75) Inventors: Antonio Nardi, Ballerup (DK); Joachim Demnitz, Kobenhavn N (DK); Morten Grunnet, Kobenhavn O (DK); Palle Christophersen, Ballerup (DK); David Spencer Jones, Smorum (DK); Elsebet Østergaard Nielsen, Kobenhavn K (DK); Dorte Strøbæk, Farum (DK); Lars Siim Madsen, Soro (DK)

Correspondence Address: BIRCH STEWART KOLASCH & BIRCH, PO BOX 747, FALLS CHURCH, VA, 22040-0747, US

(73) Assignee: Neuro Search A/S, Ballerup (DK)

(21) Appl. No.: 12/599,196

(22) PCT Filed: May 7, 2008
(86) PCT No.: PCT/EP2008/055628

§ 371 Date: Jan. 5, 2010

Related U.S. Application Data
(60) Provisional application No. 60/916,881, filed on May 9, 2007.

(30) Foreign Application Priority Data
May 8, 2007 (DK) PA 2007 00689

(51) Int. Cl. A61K 31/41; C07D 257/04; A61P 11/00; A61P 25/08; A61P 25/00; A61P 13/10; A61P 15/10; A61P 15/00; A61P 13/00; A61P 25/18; A61P 25/16; A61P 25/24; A61P 27/00; A61P 17/14

(52) U.S. Cl. 514/381; 548/254; 548/253; 548/252

(57) ABSTRACT

This invention relates to novel benzamidine derivatives of formula (I) that are found to be potent modulators of potassium channels and, as such, are valuable candidates for the treatment of diseases or disorders as diverse as those which are responsive to the modulation of potassium channels. A stereoisomer or a mixture of its stereoisomers, or an N-oxide thereof, or a pharmaceutically-acceptable addition salt thereof, wherein B may be absent (i.e. no heterocyclic ring is formed); and when absent, the nitrogen next to B holds a hydrogen (i.e. “NH”), and A represents NH₂ or OH; or B may be present (i.e. forms part of a heterocyclic ring); and when present, B represents C═O or C═S; and A represents NH or O; and wherein the rest of variables are as specified in claim 1.

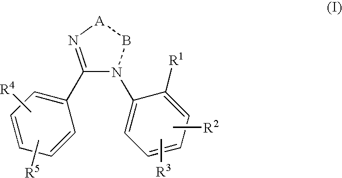

### TECHNICAL FIELD

This invention relates to novel benzamidine derivatives that are found to be potent modulators of potassium channels and, as such, are valuable candidates for the treatment of diseases or disorders as diverse as those which are responsive to the modulation of potassium channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation.

Dysfunction of potassium channels, as well as other ion channels, generates loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain, and cancer.

Among the large number of potassium channel types, the large-conductance calcium-activated potassium channel subtype is an obvious site for pharmacological intervention and for the development of new potassium channel modulators. Their physiological role has been especially studied in the nervous system, where they are key regulators of neuronal excitability and of neurotransmitter release, and in smooth muscle, where they are crucial in modulating the tone of vascular, broncho-tracheal, urethral, uterine or gastro-intestinal musculature.

Given these implications, small agents with BK-opening properties could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions, anxiety and pain. As far as the cardiovascular system is concerned, the physiological function of these ion channels represents a fundamental steady state mechanism, modulating vessel depolarisation, vasoconstriction and increases of intravascular pressure, and the development of selective activators of BK channels is seen as a potential pharmacotherapy of vascular diseases, including hypertension, erectile dysfunction, coronary diseases and vascular complications associated with diabetes or hypercholesterolemia.

WO 2006/055760 and WO 2007/139967 describe triazoles derivatives useful as HSP90 inhibitors for the treatment of e.g. cancer. However, the benzamidine derivatives of the present invention have not been described and their activity as potassium channel modulators certainly no suggested.

### SUMMARY OF THE INVENTION

It is an object of the invention to provide novel benzamidine derivatives useful as potassium channel modulators. The benzamidine derivatives of the invention may be characterised by Formula I

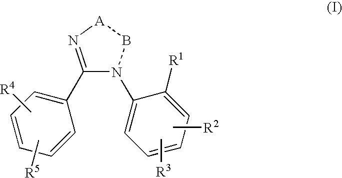

a stereoisomer or a mixture of its stereoisomers, or an N-oxide thereof, or a pharmaceutically-acceptable addition salt thereof, wherein

B may be absent (i.e. no heterocyclic ring is formed); and when absent, the nitrogen next to B holds a hydrogen (i.e. “NH”); and A represents NH₂ or OH; or

B may be present (i.e. forms part of a heterocyclic ring); and when present, B represents C═O or C═S; and A represents NH or O;

R¹ represents a tetrazolyl group;

R² and R³, independently of each other, represent hydrogen, halo, cyano, nitro, hydroxy or phenyl, which phenyl may optionally be substituted one or more times with halo, trifluoromethyl and/or hydroxy; and

R⁴ and R⁵, independently of each other, represent halo, trifluoromethyl, hydroxy and/or phenyl.

In another aspect the invention provides pharmaceutical compositions comprising a therapeutically effective amount of a benzamidine derivative of the invention.

In a third aspect the invention relates to the use of the benzamidine derivatives of the invention for the manufacture of pharmaceutical compositions.

In a further aspect the invention provides a method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the benzamidine derivative of the invention.

Other objects of the invention will be apparent to the person skilled in the art from the following detailed description and examples.

### DETAILED DISCLOSURE OF THE INVENTION

In its first aspect the invention provides novel benzamidine derivatives of Formula I

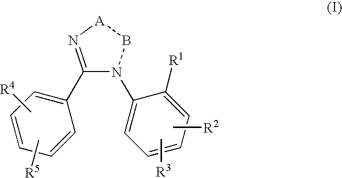

a stereoisomer or a mixture of its stereoisomers, or an N-oxide thereof, or a pharmaceutically-acceptable addition salt thereof, wherein

B may be absent (i.e. no heterocyclic ring is formed); and when absent, the nitrogen next to B holds a hydrogen (i.e. “NH”); and A represents NH₂ or OH; or

B may be present (i.e. forms part of a heterocyclic ring); and when present, B represents C═O or C═S; and A represents NH or O;

R¹ represents a tetrazolyl group;

R² and R³, independently of each other, represent hydrogen, halo, cyano, nitro, hydroxy or phenyl, which phenyl may optionally be substituted one or more times with halo, trifluoromethyl and/or hydroxy; and

R⁴ and R⁵, independently of each other, represent halo, trifluoromethyl, hydroxy and/or phenyl.

In a preferred embodiment the benzamidine derivatives of the invention is a compound of Formula I, a stereoisomer or a mixture of its stereoisomers, or a pharmaceutically-acceptable addition salt thereof, wherein

A represents a nitrogen (N) or an oxygen (O) atom;

B is absent (i.e. no heterocyclic ring is formed) or is present (i.e. forms part of a heterocyclic ring); and when present, B represents C═O or C═S;

R¹ represents a tetrazolyl group;

R² and R³, independently of each other, represent hydrogen, halo, cyano, nitro, hydroxy or phenyl, which phenyl may optionally be substituted one or more times with halo, trifluoromethyl and/or hydroxy; and

R⁴ and R⁵, independently of each other, represent halo, trifluoromethyl, hydroxy and/or phenyl.

In another preferred embodiment, the benzamidine derivative of the invention is a compound of Formula Ia

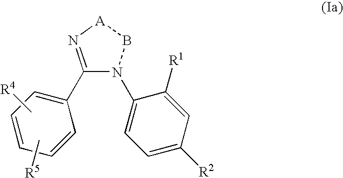

a stereoisomer or a mixture of its stereoisomers, or an N-oxide thereof, or a pharmaceutically-acceptable addition salt thereof, wherein

B may be absent (i.e. no heterocyclic ring is formed); and when absent, the nitrogen next to B holds a hydrogen (i.e. “NH”); and A represents NH₂ or OH; or

B may be present (i.e. forms part of a heterocyclic ring); and when present, B represents C═O or C═S; and A represents NH or O; and R¹, R², R⁴ and R⁵ are as defined above, (i.e. R³ represent hydrogen).

In a third preferred embodiment, the benzamidine derivative of the invention is a compound of Formula Ia a stereoisomer or a mixture of its stereoisomers, or a pharmaceutically-acceptable addition salt thereof, wherein A, B, R¹, R², R⁴ and R⁵ are as defined above, (i.e. R³ represent hydrogen).

In a fourth preferred embodiment, the benzamidine derivative of the invention is a compound of Formula Ib

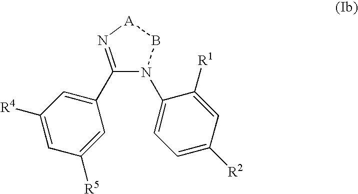

a stereoisomer or a mixture of its stereoisomers, or an N-oxide thereof, or a pharmaceutically-acceptable addition salt thereof, wherein

B may be absent (i.e. no heterocyclic ring is formed); and when absent, the nitrogen next to B holds a hydrogen (i.e. “NH”); and A represents NH₂ or OH; or

B may be present (i.e. forms part of a heterocyclic ring); and when present, B represents C═O or C═S; and A represents NH or O; and

R¹, R², R⁴ and R⁵ are as defined above (i.e. R³ represent hydrogen).

In a fifth preferred embodiment, the benzamidine derivative of the invention is a compound of Formula Iba stereoisomer or a mixture of its stereoisomers, or a pharmaceutically-acceptable addition salt thereof, wherein A, B, R¹, R², R⁴ and R⁵ are as defined above (i.e. R³ represent hydrogen).

In a sixth preferred embodiment, the benzamidine derivative of the invention is a compound of Formula Ic

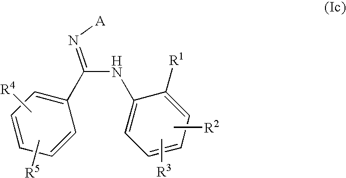

a stereoisomer or a mixture of its stereoisomers, or an N-oxide thereof, or a pharmaceutically-acceptable addition salt thereof, wherein

A represents NH2 or OH; and

R¹, R², R³, R⁴ and R⁵ are as defined above.

In a seventh preferred embodiment, the benzamidine derivative of the invention is a compound of Formula Ica stereoisomer or a mixture of its stereoisomers, or a pharmaceutically-acceptable addition salt thereof, wherein A, R¹, R², R³, R⁴ and R⁵ are as defined above.

In an eighth preferred embodiment, the benzamidine derivative of the invention is a compound of Formula I, Ia or Ib, or an N-oxide thereof, or a pharmaceutically-acceptable addition salt thereof, wherein

B is be absent (i.e. no heterocyclic ring is formed); and the nitrogen next to B holds a hydrogen (i.e. “NH”), and

A represents NH₂ or OH.

In a more preferred embodiment, the benzamidine derivative of the invention is a compound of Formula I, Ia, Ib or Ic, or an N-oxide thereof, or a pharmaceutically-acceptable addition salt thereof, wherein A represents a nitrogen (N) or an oxygen (O) atom.

In another more preferred embodiment, A represents a nitrogen (N) atom.

In a third more preferred embodiment, A represents an oxygen (O) atom.

In a fourth more preferred embodiment, A represents NH₂.

In a fifth more preferred embodiment, A represents OH.

In a ninth preferred embodiment, the benzamidine derivative of the invention is a compound of Formula I, Ia or Ib, or an N-oxide thereof, or a pharmaceutically-acceptable addition salt thereof, wherein B is present (i.e. forms part of a heterocyclic ring) and represents C═O or C═S; and A represents NH or O.

In a more preferred embodiment, B is represents C═O; and A represents NH or O.

In another more preferred embodiment, B is represents C═O; and A represents NH.

In a third more preferred embodiment, B is represents C═O; and A represents O.

In a fourth more preferred embodiment, B is represents C═S; and A represents NH or O.

In a fifth more preferred embodiment, B is represents C═S; and A represents NH.

In a sixth more preferred embodiment, B is represents C═S; and A represents O.

In a tenth preferred embodiment, the benzamidine derivative of the invention is a compound of Formula I, Ia or Ib, or an N-oxide thereof, or a pharmaceutically-acceptable addition salt thereof, wherein B is absent (i.e. no heterocyclic ring is formed), or B is present (i.e. forms part of a heterocyclic ring); and when present, B represents C═O or C═S.

In a more preferred embodiment B is present (i.e. forms part of a heterocyclic ring); and presents C═O or C═S.

In another more preferred embodiment B is present (i.e. forms part of a heterocyclic ring); and presents C═O.

In a third more preferred embodiment B is present (i.e. forms part of a heterocyclic ring); and presents C═S.

In a fourth more preferred embodiment, the benzamidine derivative of the invention is a compound of Formula Ic, i.e. B is absent (and no heterocyclic ring is formed).

In an eleventh preferred embodiment, the benzamidine derivative of the invention is a compound of Formula I, Ia, Ib or Ic, or an N-oxide thereof, or a pharmaceutically-acceptable addition salt thereof, wherein R¹ represents a tetrazolyl group.

In a more preferred embodiment R¹ represents a 1H-tetrazol-5-yl or 2H-tetrazol-5-yl group.

In another more preferred embodiment R¹ represents a 1H-tetrazol-5-yl group.

In a third more preferred embodiment R¹ represents a 2H-tetrazol-5-yl group.

In a twelfth preferred embodiment, the benzamidine derivative of the invention is a compound of Formula I, Ia, Ib or Ic, or an N-oxide thereof, or a pharmaceutically-acceptable addition salt thereof, wherein R² and R³, independently of each other, represent hydrogen, halo, cyano, nitro, hydroxy or phenyl, which phenyl may optionally be substituted one or more times with halo, trifluoromethyl and/or hydroxy.

In a more preferred embodiment R² and R³, independently of each other, represent hydrogen or phenyl, which phenyl may optionally be substituted one or more times with halo, trifluoromethyl and/or hydroxy.

In another more preferred embodiment R² represents phenyl, which phenyl may optionally be substituted one or more times with halo, trifluoromethyl and/or hydroxy; and R³ represents hydrogen.

In a third more preferred embodiment R² represents phenyl substituted with halo, trifluoromethyl or hydroxy; and R³ represents hydrogen.

In a fourth more preferred embodiment R² represents phenyl substituted with trifluoromethyl; and R³ represents hydrogen.

In a thirteenth preferred embodiment, the benzamidine derivative of the invention is a compound of Formula I, Ia, Ib or Ic, or an N-oxide thereof, or a pharmaceutically-acceptable addition salt thereof, wherein R⁴ and R⁵, independently of each other, represent halo, trifluoromethyl, hydroxy and/or phenyl.

In a more preferred embodiment R⁴ and R⁵, independently of each other, represent halo, trifluoromethyl and/or phenyl.

In another more preferred embodiment R⁴ and R⁵, independently of each other, represent halo or trifluoromethyl.

In a third more preferred embodiment R⁴ and R⁵ both represent halo, trifluoromethyl or phenyl.

In a fourth more preferred embodiment R⁴ and R⁵ both represent halo, trifluoromethyl, hydroxy and/or phenyl.

In a fifth more preferred embodiment R⁴ and R⁵ both represent halo or trifluoromethyl.

In a sixth more preferred embodiment R⁴ and R⁵ both represent trifluoromethyl.

In a most preferred embodiment the benzamidine derivative of the invention is

N-Amino-N′-[3-(1H-tetrazol-5-yl)-4′-trifluoromethyl-biphenyl-4-yl]-3,5-bis-trifluoromethyl-benzamidine;

5-(3,5-Bis-trifluoromethyl-phenyl)-4-[3-(2H-tetrazol-5-yl)-4′-trifluoromethyl-biphenyl-4-yl]-2,4-dihydro-[1,2,4]triazol-3-one; or

N-Hydroxy-N′-[3-(1H-tetrazol-5-yl)-4′-trifluoromethyl-biphenyl-4-yl]-3,5-bis-trifluoromethyl-benzamidine;

or a pharmaceutically-acceptable addition salt thereof.

Any combination of two or more of the embodiments described herein is considered within the scope of the present invention.

### Definition of Substituents

In the context of this invention halo represents fluoro, chloro, bromo or iodo.

### Pharmaceutically Acceptable Salts

The benzamidine derivatives of the invention may be provided in any form suitable for the intended administration. Suitable forms include pharmaceutically (i.e. physiologically) acceptable salts, and pre- or prodrug forms of the benzamidine derivative of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a benzamidine derivative of the invention include, without limitation, the sodium, the potassium, the calcium, the magnesium, the lithium, and the ammonium salt, and the like, of a benzamidine derivative of the invention containing an anionic group. Such cationic salts may be formed by procedures well known and described in the art.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such isomers and any mixtures thereof including racemic mixtures.

Racemic forms can be resolved into the optical antipodes by known methods and techniques. One way of resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Preparation

The compounds according to the invention may be prepared by conventional methods for chemical synthesis, e.g. those described in the working examples.

### Biological Activity

The benzamidine derivatives of the invention have been found to possess potassium channel modulating activity as measured by standard electrophysiological methods. Due to their activity at the potassium channels, the benzamidine derivatives of the invention are considered useful for the treatment of a wide range of diseases and conditions.

In a special embodiment, the benzamidine derivatives of the invention are considered useful for the treatment, prevention or alleviation of a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjogren's syndrome, xerostomia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma) or baldness.

In a more preferred embodiment, the benzamidine derivatives of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the benzamidine derivatives of the invention are considered useful for the treatment, prevention or alleviation of psychosis, schizophrenia, bipolar disorder, depression, epilepsy, Parkinson's disease or pain.

In a third more preferred embodiment, the benzamidine derivatives of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the benzamidine derivatives of the invention are considered useful for the treatment, prevention or alleviation of cardiac ischemia, ischemic heart disease, hypertrophic heart, cardiomyopathy or failing heart.

In a fifth more preferred embodiment, the compounds of the invention are considered useful for the treatment, prevention or alleviation of a cardiovascular disease. In a more preferred embodiment the cardiovascular disease is atherosclerosis, ischemia/reperfusion, hypertension, restenosis, arterial inflammation, myocardial ischaemia or ischaemic heart disease.

In an sixth more preferred embodiment, the compounds of the invention are considered useful for obtaining preconditioning of the heart. Preconditioning, which includes ischemic preconditioning and myocardial preconditioning, describes short periods of ischemic events before initiation of a long lasting ischemia. The compounds of the invention are believed having an effect similar to preconditioning obtained by such ischemic events. Preconditioning protects against later tissue damage resulting from the long lasting ischemic events.

In a seventh more preferred embodiment, the benzamidine derivatives of the invention are considered useful for the treatment, prevention or alleviation of schizophrenia, depression or Parkinson's disease.

In an eight more preferred embodiment, the compounds of the invention are considered useful for the treatment, prevention or alleviation of an obstructive or inflammatory airway disease. In a more preferred embodiment the the obstructive or inflammatory airway disease is an airway hyperreactivity, a pneumoconiosis such as aluminosis, anthracosis, asbestosis, chalicosis, ptilosis, siderosis, silicosis, tabacosis and byssinosis, a chronic obstructive pulmonary disease (COPD), bronchitis, excerbation of airways hyperreactivity or cystic fibrosis.

In its most preferred embodiment the obstructive airway disease is chronic obstructive pulmonary disease (COPD).

In a ninth more preferred embodiment, the benzamidine derivatives of the invention are considered useful for the treatment, prevention or alleviation of a sexual dysfunction, incl. male sexual dysfunction and female sexual dysfunction, and incl. male erectile dysfunction.

In an even more preferred embodiment the benzamidine derivative of the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the benzamidine derivative of the invention is used in a combination therapy together with sildenafil, tadalafil, vardenafil or calcium dobesilate.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred benzamidine derivatives of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of a benzamidine derivative of the invention.

While a benzamidine derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the benzamidine derivative of the invention together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Pharmaceutical Kits of Parts

According to the invention there is also provided a kit of parts comprising at least two separate unit dosage forms (A) and (B):

(A) a benzamidine derivative of the invention; and

(B1) a phosphodiesterase inhibitor; or

(B2) an agent that potentiates endothelium-derived hyperpolarizing factor-mediated responses; and optionally

(C) instructions for the simultaneous, sequential or separate administration of the benzamidine derivative of A, and the phosphodiesterase inhibitor of B1, or an agent that potentiates endothelium-derived hyperpolarizing factor-mediated responses of B2, to a patient in need thereof.

In a more preferred embodiment the phosphodiesterase inhibitor for use according to the invention (B1) is a phosphodiesterase 5 (PDE5) inhibitor, and in an even more preferred embodiment the phosphodiesterase inhibitor for use according to the invention is sildenafil, tadalafil or vardenafil.

In another more preferred embodiment the agent that potentiates endothelium-derived hyperpolarizing factor-mediated responses for use according to the invention (B2) is calcium dobesilate.

The benzamidine derivative of the invention and the phosphodiesterase inhibitor or the agent that potentiates endothelium-derived hyperpolarizing factor-mediated responses for use according to the invention may preferably be provided in a form that is suitable for administration in conjunction with the other. This is intended to include instances where one or the other of two formulations may be administered (optionally repeatedly) prior to, after, and/or at the same time as administration with the other component.

Also, the benzamidine derivative of the invention and the phosphodiesterase inhibitor or the agent that potentiates endothelium-derived hyperpolarizing factor-mediated responses for use according to the invention may be administered in a combined form, or separately or separately and sequentially, wherein the sequential administration is close in time or remote in time. This may in particular include that two formulations are administered (optionally repeatedly) sufficiently closely in time for there to be a beneficial effect for the patient, that is greater over the course of the treatment of the relevant condition than if either of the two formulations are administered (optionally repeatedly) alone, in the absence of the other formulation, over the same course of treatment. Determination of whether a combination provides a greater beneficial effect in respect of, and over the course of treatment of, a particular condition, will depend upon the condition to be treated or prevented, but may be achieved routinely by the person skilled in the art.

When used in this context, the terms “administered simultaneously” and “administered at the same time as” include that individual doses of the positive allosteric nicotine receptor modulator and the cognitive enhancer are administered within 48 hours, e.g. 24 hours, of each other.

Bringing the two components into association with each other, includes that components (A) and (B) may be provided as separate formulations (i.e. independently of one another), which are subsequently brought together for use in conjunction with each other in combination therapy; or packaged and presented together as separate components of a “combination pack” for use in conjunction with each other in combination therapy.

### Methods of Therapy

In another aspect the invention provides a method of treatment, prevention or alleviation of a disease, disorder or condition of a living animal body, including a human, which disorder, disease or condition is responsive to activation of a potassium channel, which method comprises the step of administering to such a living animal body in need thereof, a therapeutically effective amount a compound capable of activating the potassium channel, or a pharmaceutically-acceptable addition salt thereof.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Preparatory Example

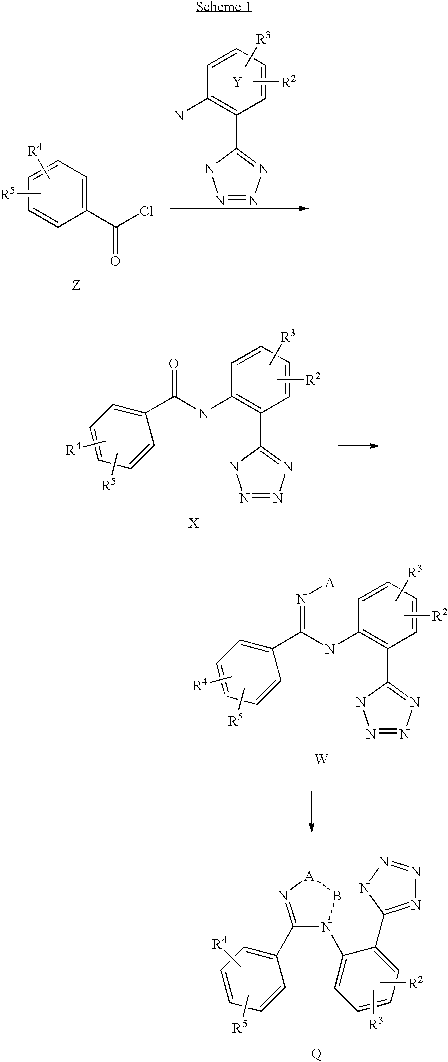

The synthetic pathway of the compounds of the invention is outlined in Scheme 1. It involves coupling between the suitable benzoylchlorides (Z), commercially available or prepared from the corresponding commercial benzoic acids by treatment with thionyl chloride, and a number of 2-(1H-tetrazol-5-yl)-phenylamines (Y), to afford the correspondent benzamide intermediates X. The aniline derivatives Y were either synthesised as previously described in e.g. WO 98/47879 and in Valgeirsson et al. in Journal of Medicinal Chemistry 2004 47 (27) 6948-6957, or may easily synthesised by palladium catalyzed Suzuki cross-coupling reaction between cyano-substituted aryl halides and aryl boronic acids, followed by the conversion of the resulting cyano derivative to correspondent tetrazolyl derivative.

As an example of this latter experimental procedure, the synthesis of the aniline intermediates C and D are reported in Scheme 2. As example of synthesis of benzanilides X, the experimental procedure of the novel intermediate E is reported. Benzanilides X were then treated with phosphorus pentachloride in dry toluene (˜100° C.) to generate the correspondent iminoyl chlorides (as described by Eloy F et al. in European Journal of Medicinal Chemistry 1974 9 (6) 602-6), followed by reaction with a solution of anhydrous hydrazine or hydroxylamine, to yield the correspondent carbohydradrazonamides or amidoximes (W). The resulting carbohydradrazonamides or amidoximes finally undergoes a ring closure with or 1,1′-thiocarbonyldiimidazole, CDI or triphosgene, to afford derivatives Q described in Scheme 1.

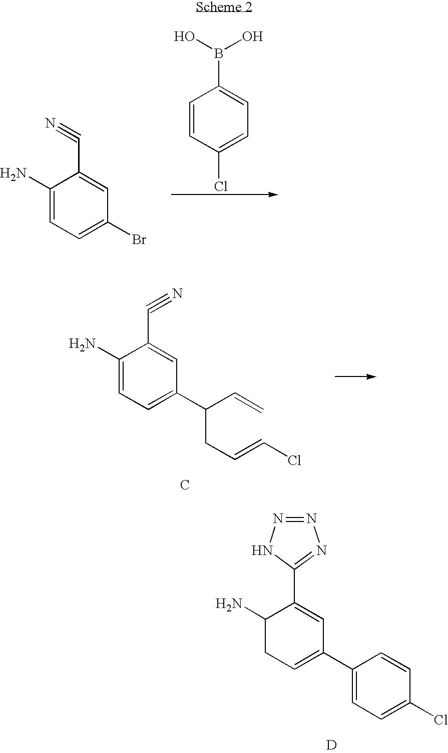

### 4-Amino-4′-chloro-biphenyl-3-carbonitrile (Intermediate Compound) (C)

To a mixture of the commercial 2-amino-5-bromo-benzonitrile (5.5 g, 1 eq), 4-chlorobenzeneboronic acid (4.8 g, 1.1 eq), potassium carbonate (12.7 g, 3.3 eq), dimethoxy ethane (80 ml) and water (40 ml), bistriphenylphosphine palladium (II) chloride (0.2 g) is added. The resulting mixture is refluxed for 24 hours and then evaporated to dryness. The residue is purified by flash chromatography using dichloromethane as eluent (5.32 g, 83% yield).

### 4′-Chloro-3-(1H-tetrazol-5-yl)-biphenyl-4-ylamine (Intermediate Compound) (D)

A mixture of 4-amino-4′-chloro-biphenyl-3-carbonitrile (5.3 g, 1 eq), sodium azide (2.3 g, 1.5 eq) and trethylamine hydrochloride (4.9 g, 1.5 eq) is suspended in 40 ml of toluene and heated (60° C.) overnight. To the reaction mixture, cooled to room temperature, water and 4M HCl are added, to afford the title compound as a white precipitate. This was collected by filtration (4.83 g, 77% yield) and used for the next step without further purification.

### N-[3-(1H-tetrazol-5-yl)-4′-trifluoromethyl-biphenyl-4-yl]-3,5-bis-trifluoromethyl-benzamide (Intermediate Compound) (E)

To a stirred solution of 3-(1 H-tetrazol-5-yl)-4′-trifluoromethyl-biphenyl-4-ylamine (0.25 g) (prepared as described in US 2002037905) in pyridine (4 ml), commercial 3,5-Bis(trifluoromethyl)benzoylchloride (0.226 g, 1 eq) is added portion wise and stirring is continued at room temperature for 12 hours. The resulting reaction mixture is evaporated to dryness and the solid residue is washed with HCl 1N and water, and used as such for the next reaction (0.41, yield 91%). LC-ESI-HRMS of [M−H]− shows 544.0804 Da. Calc. 544.081987 Da, dev. −2.9 ppm.

### N-Amino-N′-[3-(1H-tetrazol-5-yl)-4′-trifluoromethyl-biphenyl-4-yl]-3,5-bis-trifluoromethyl-benzamidine (1)

To a stirred solution of intermediate E (0.4 g) in dry toluene (25 ml) and dry methylene chloride (3 ml), kept under nitrogen, phosphorus pentachloride (0.76 g, 5 eq) is added and the mixture refluxed for 20 hours. The reaction mixture is then evaporated to dryness and the residue is taken up in dry tetrahydrofuran (40 mL) and cooled to −10° C. To this, a commercial solution of hydrazine 1M in tetrahydrofuran (7.3 ml, 10 eq) is added and the reaction mixture is allowed to reach room temperature spontaneously and further stirred for 3 hours at room temperature. Evaporation to dryness of the new reaction mixture provided a yellow oil, which crystallized upon treatment with 5 ml of petroleum ether 40-60 (0.39 g, yield 95%). This crude solid was easily purified by crystallization from methylene chloride/petroleum ether (40-60). LC-ESI-HRMS of [M−H]− shows 558.1085 Da. Calc. 558.10887 Da, dev. −0.7 ppm.

### 5-(3,5-Bis-trifluoromethyl-phenyl)-4-[3-(2H-tetrazol-5-yl)-4′-trifluoromethyl-biphenyl-4-yl]-2,4-dihydro-[1,2,4]triazol-3-one (2)

To a stirred solution of the compound 1 (0.35 g) in dry pyridine (3 ml), triphosgene (0.111 g, 0.6 eq) is added and stirring is continued at room temperature for 20 min. The solution is heated at 70° C. for 5 hours and the pyridine solution is purified by prep-LCMS, to provide the title compound as a white solid (0.04 g, yield 11%). ¹H-NMR (DMSO-d₆): 12.43 (1H, s, NH), 8.41-7.60 (10H, m, aromatics).

### N-Hydroxy-N′-[3-(1H-tetrazol-5-yl)-4′-trifluoromethyl-biphenyl-4-yl]-3,5-bis-trifluoromethyl-benzamidine (3)

To a stirred solution of intermediate E (1.00 g) in dry toluene (40 ml) and dry methylene chloride (6 ml), kept under nitrogen, phosphorus pentachloride (1.9 g, 5 eq) is added and the mixture refluxed for 20 hours. The reaction mixture is then evaporated to dryness and the oil residue is taken up in dry tetrahydrofuran (15 ml) and cannulated dropwise in a stirred solution of an excess of hydroxylamine in absolute ethanol (50 ml), under nitrogen and at O° C. This latter is prepared mixing sodium methoxide (0.99 g) and hydroxylamine hydrochloride (1.27 g) in hot absolute ethanol and by filtering off the inorganic material precipitated. The reaction mixture is stirred at room temperature for 20 hours and then evaporated to dryness. The resulting solid residue is purified by prep-LCMS, to afford the title compound as yellowish solid (0.48 g yield˜47%). LC-ESI-HRMS of [M−H]− shows 559.0917 Da. Calc. 559.092886 Da, dev. −2.1 ppm.

### Example 2

### Biological Activity

### Electrophysiological Determination

In this experiment we investigated the influence of the compounds of the invention on the membrane currents when determined electrophysiologically on Xenopus Oocytes capable of expressing human BK channels, and the current through the channels is recorded using the classical two-electrode voltage clamp technique.

The results of these determinations are presented in Table 1 below, and presented as the increase of BK current relative to the basal current.

TABLE 1
Influence on BK Current

| | | Relative Increase of |
|---|---|---|
| Compound | Concentration | BK Current |
| 1 | 30 μM | 1118% |
| 2 | 30 μM | 252% |
| 3 | 30 μM | 1342% |

1-11. (canceled)

12. A benzamidine derivative of Formula I 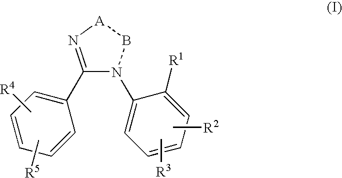
a stereoisomer or a mixture of its stereoisomers, or an N-oxide thereof, or a pharmaceutically-acceptable addition salt thereof, wherein
B may be absent (i.e. no heterocyclic ring is formed); and when absent, the nitrogen next to B holds a hydrogen (i.e. “NH”), and A represents NH₂ or OH; or
B may be present (i.e. forms part of a heterocyclic ring); and when present, B represents C═O or C═S; and A represents NH or O;
R¹ represents a tetrazolyl group;
R² and R³, independently of each other, represent hydrogen, halo, cyano, nitro, hydroxy or phenyl, which phenyl may optionally be substituted one or more times with halo, trifluoromethyl and/or hydroxy; and
R⁴ and R⁵, independently of each other, represent halo, trifluoromethyl, hydroxy and/or phenyl.

13. The benzamidine derivative of claim 12, a stereoisomer or a mixture of its stereoisomers, or an N-oxide thereof, or a pharmaceutically-acceptable addition salt thereof, wherein
B is be absent (i.e. no heterocyclic ring is formed); and the nitrogen next to B holds a hydrogen (i.e. “NH”); and
A represents NH, or OH.

14. The benzamidine derivative of claim 12, a stereoisomer or a mixture of its stereoisomers, or an N-oxide thereof, or a pharmaceutically-acceptable addition salt thereof, wherein
B is present (i.e. forms part of a heterocyclic ring) and represents C═O or C═S; and
A represents NH or O.

15. The benzamidine derivative of claim 12, a stereoisomer or a mixture of its stereoisomers, or an N-oxide thereof, or a pharmaceutically-acceptable addition salt thereof, wherein R¹ represents a tetrazolyl group.

16. The benzamidine derivative of claim 12, a stereoisomer or a mixture of its stereoisomers, or an N-oxide thereof, or a pharmaceutically-acceptable addition salt thereof, wherein R² and R³, independently of each other, represent hydrogen, halo, cyano, nitro, hydroxy or phenyl, which phenyl may optionally be substituted one or more times with halo, trifluoromethyl and/or hydroxy.

17. The benzamidine derivative of claim 12, a stereoisomer or a mixture of its stereoisomers, or an N-oxide thereof, or a pharmaceutically-acceptable addition salt thereof, wherein R⁴ and R⁵, independently of each other, represent halo, trifluoromethyl, hydroxy and/or phenyl.

18. The benzamidine derivative of claim 12, which is
N-Amino-N′-[3-(1H-tetrazol-5-yl)-4′-trifluoromethyl-biphenyl-4-yl]-3,5-bis-trifluoromethyl-benzamidine;
5-(3,5-Bis-trifluoromethyl-phenyl)-4-[3-(2H-tetrazol-5-yl)-4′-trifluoromethyl-biphenyl-4-yl]-2,4-dihydro-[1,2,4]triazol-3-one; or
N-Hydroxy-N′-[3-(1H-tetrazol-5-yl)-4′-trifluoromethyl-biphenyl-4-yl]-3,5-bis-trifluoromethyl-benzamidine;
a stereoisomer or a mixture of its stereoisomers, or an N-oxide thereof, or a pharmaceutically-acceptable addition salt thereof.

19. A pharmaceutical composition comprising a therapeutically effective amount of the benzamidine derivative of claim 12, a stereoisomer or a mixture of its stereoisomers, or an N-oxide thereof, or a pharmaceutically-acceptable addition salt thereof, together with one or more adjuvants, excipients, carriers and/or diluents.

20. A method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the benzamidine derivative according to claim 12, a stereoisomer or a mixture of its stereoisomers, or an N-oxide thereof, or a pharmaceutically-acceptable addition salt thereof

21. The method according to claim 20, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjogren's syndrome, xerostomia, cardiovascular disorders, hypertension, myotonic dystrophy, myotonic muscle dystrophia, spasticity, xerostomi, diabetes Type II, hyperinsulinemia, premature labour, cancer, brain tumors, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma) or baldness.
